(19) United States
(12) Patent Application Publication
(10) Pub. No.: US 2011/0311642 A1
(43) Pub. Date: Dec. 22, 2011

(54) COMPOSITION AND METHOD FOR ORAL DELIVERY OF COBRA VENOM

(76) Inventor: Paul Reid, Sunrise, FL (US)

(21) Appl. No.: 13/044,512

(22) Filed: Mar. 9, 2011

Related U.S. Application Data
(63) Continuation of application No. 12/849,679, filed on Aug. 3, 2010, now abandoned.
(60) Provisional application No. 61/273,314, filed on Aug. 3, 2009.

(51) Int. Cl. A61K 35/58; A61P 29/00

(52) U.S. Cl. 424/542

(57) ABSTRACT

A composition of sterile cobra venom and a method for its oral administration to provide significant analgesic effects to a human and/or animal are disclosed. Such cobra venom compositions comprise a sterilized solution preserved by the addition of one or more suitable food-grade preservatives. The venom composition may be conveniently administered orally by means of a metered spray device.

### CROSS-REFERENCE TO RELATED APPLICATIONS

This application is a continuation application from earlier-filed U.S. patent application Ser. No. 12/849,679 filed on Aug. 3, 2010, which claimed the benefit of priority of Provisional Patent Application No. 61/273,314, filed Aug. 3, 2009.

### FIELD OF THE INVENTION

This invention relates generally to the field of pharmaceutical and healthcare products for the treatment of pain, and more particularly to formulations of sterile cobra venom suitable for oral administration, and products comprising these formulations in liquid and spray forms.

### BACKGROUND OF THE INVENTION

Millions of people around the world suffer from untreated pain related to a variety of illnesses and ailments, as well as from unidentified causes. Humans have searched for effective painkillers for many, many years. Natural pain-killing compositions have been discovered from various sources as varied as willow bark, opium poppies (a source of morphine, codeine, and thebaine), and snake venoms. Opium, for example, was used as a narcotic by Hippocrates, introduced to Persia and India by Alexander the Great, and used as a painkiller by Paracelsus during the Renaissance.

Despite their effectiveness as analgesics, opiate drugs such as morphine and codeine are classified as narcotics and their use is subject to complex legal and medical regulations in most countries. Furthermore, opiate drugs have a high potential for addiction and abuse.

Clinical investigations from the 1930's through the 1950's revealed that cobra venom is a potent pain killer with activity superior to morphine, but without the known adverse effects of opiates. In the United States, cobra venom for medicinal use is only available in the form of homeopathic products that contain extremely low concentrations of the active product. The use of medicinal products in homeopathy centers on the logarithmic dilution of the active product to a specific point where it is then deemed ready for use. These dilutions are so high that they may result in the absence of the original product in the formulation administered to the patient. In homeopathy, the preferred dilution of cobra venom is a 1:10,000 dilution, although the volume actually administered has been quite variable over the years. In 1870's Europe, the preferred dose was 10⁻⁴ (1:10,000 dilution). Present guidelines, as provided in the Homeopathic Pharmacopoeia of the United States (USHP), list the recommended dilutions in the range of 10⁻⁶ to 10⁻⁸.

In Chinese medicine, the venom is prepared on demand and small quantities, usually sufficient for one week, are given to the patient. Alternatively, the dried venom is mixed into lactose (triturated) and provided as small pills. The venom solution is then mixed with tea or water. The dosage to be used is left to the discretion of the treating physician.

Unfortunately, at these low dilutions, the direct ingestion of cobra venom left subjects with unpleasant side effects that included irritated and sore throat, headache, nausea, vomiting, abdominal cramps and pain, sudden bowel movements and diarrhea. Given the existence of such problematic side effects, not surprisingly the utilization of oral cobra venom as a pain remedy declined, and was ultimately abandoned in Western medicine.

The use of cobra venom as a treatment for pain enjoyed a short resurgence in the 1930's following research and clinical studies that revealed that cobra venom had very potent analgesic activity. However, during this period cobra venom was administered only by injection, requiring that the venom solution be rendered sterile prior to use. This was accomplished by prolonged exposure of the venom to heat in the range of 60° Celsius. While clinically successful and safe, it required frequent injections of cobra venom by physicians and this method of administration also fell out of favor by the 1970's.

Considering the favorable outcomes related to pain relief reported for injectable cobra venom in trials conducted during the 1930's to the 1970's, although associated with side-effects, and considering the problems associated with opiate-based medications, what are needed are compositions and methods for providing analgesic levels of cobra venom in an orally-administered form suitable for self-administration by a patient in need of the substantial pain relief that cobra venom may provide and free of the side-effects which have previously limited its use.

### SUMMARY OF THE INVENTION

The present invention relates to a novel oral formulation of cobra venom, and methods for the oral administration of cobra venom. More particularly, the invention provides formulations of sterile solutions of cobra venom containing a preservative that are suitable for oral administration in several forms, including as beverages and oral sprays that can be used with an oral delivery device to permit convenient, metered administration of the venom. The resulting solutions and delivery systems are safe for the storage and administration of cobra venom over extended periods of time.

In one aspect, the invention provides a composition of cobra venom suitable for oral administration. Such a composition comprises a sterile cobra venom solution admixed with a food-grade preservative. In various aspects, the preservative may be chosen from among the group consisting of methyl paraben, sodium benzoate, potassium sorbate, and combinations thereof.

In various aspects of the invention, a sterile cobra venom solution may be formulated at a final homeopathic concentration of 3×, 4×, and/or 5×. Such homeopathic concentrations generally may contain from about 0.035 to about 0.35 mg/ml, from about 0.0035 to about 0.035 mg/ml, and from about 0.00035 to about 0.0035 mg/ml of venom protein, respectively.

In another aspect, the invention provides a method for the oral administration of a composition comprising a sterile cobra venom solution and a food-grade preservative, the method comprising administering the composition as a spray or jet. Some aspects of the invention provide a healthcare product comprising a solution of sterile cobra venom admixed with a food-grade preservative, the venom in the solution having a homeopathic formulation of from about 3× to about 5×, and a metered pump configured to deliver a volume of the solution in the range of from about 0.05 to about 1 ml.

In various aspects of the invention, a composition is provided as a beverage. Compositions may also be provided for release from edible films, for example, which may be placed on the mucosa within the mouth.

### DETAILED DESCRIPTION OF THE INVENTION

The present invention relates to an oral formulation of cobra venom, and methods for the oral administration of cobra venom. More particularly, the invention provides formulations of sterile solutions of cobra venom admixed with one or more food-grade preservative(s), the combination of venom and preservative(s) being suitable for oral administration in several forms, including, for example, as beverages, oral sprays, lozenges, and edible films that can be used with an oral delivery device to permit convenient, metered administration of the venom. The resulting solutions and delivery systems provide safe storage and administration of cobra venom over extended periods of time.

Formulations of the product as described and claimed herein have been determined to be effective for the reduction of chronic pain symptoms, such as chronic back pain, in human subjects. These analgesic benefits are delivered without significant adverse effects and without potential for addiction, representing a significant advance over opioid-based analgesics.

Homeopathic cobra venom preparations may be made from the venom of the Asian cobra (e.g., Naja tripudians) and related species according to methods provided in the United States and European Homeopathic Pharmacopoeias. Historically, cobra venoms were selected by homeopaths based upon their neurotoxic activity for treatment of disorders of the nervous system. Without intending to be bound by theory, it is therefore believed that the principal active components in the venoms are most likely neurotoxins. In contrast to the opioid drugs which bind to opioid receptors (G-protein coupled receptors acting by GABAnergic neurotransmission), the venom neurotoxins are known to primarily target the cholinergic system by blocking the activity of acetylcholine, although it is possible that other receptors or targets may be involved in the analgesic effect.

Preparing an injectable form of a cobra venom solution is now straightforward. However, developing a convenient and effective oral venom formulation that avoided the known problems associated with oral administration presented several challenges. Toxicology studies in mice were conducted by the inventor, from which it was determined that mice could drink a 1 mg/ml solution of cobra venom for 28 days with a daily intake of 350 mg/kg. By contrast, injection of merely 10-12 micrograms was a fatal dose. The mice in this study gained weight, were quite active, and were apparently unaffected by the ingestion of cobra venom at this concentration via their drinking water. This perplexing and unexpected finding led the inventor to question why the side effects of oral administration of cobra venom as described in the prior art from the last century, being so to detrimental in humans, were seemingly absent in his studies with the mice.

Without intending to be bound by theory, it may be that the clinical tests on humans described in the homeopathic literature, which were conducted with orally administered cobra venom, were flawed because the venom product may have been contaminated with bacteria. Furthermore, the dosing of the material employed a concentration that was too high. Today, it is possible to remove such bacterial contamination by sterile filtration without compromising the quality of the venom. When cobra venom is prepared under sterile conditions, no abdominal problems are experienced. Sterile filtration may be accomplished, for example, using a filter having a pore size of 0.45 μm or smaller. Sterilization may also be accomplished by prolonged low-heat treatment or by pasteurization.

Providing the sterile composition for oral administration is facilitated by the addition of a food-grade preservative to the venom (i.e., at least one food-grade preservative). The inventor has determined that the use of such a preservative has no adverse effect on the efficacy of the venom composition for achieving analgesia after oral administration. Suitable food-grade preservatives include, for example, methyl paraben, sodium benzoate, and potassium sorbate, with the use of other suitable preservatives being known to those of skill in the art and within the scope of the present invention, given the present disclosure.

Throat irritation had previously been consistently reported when cobra venom was taken by mouth. While homeopathic rules guide the dilution strength of the product from the “mother tincture” (in this case raw venom), the dose that is applied has been left to the discretion of the homeopath. In the 1873 copy of Guy's Hospital Report, Taylor reported that he received 4.2 grains (273 mg) of dried cobra venom and, allowing for the loss of water through desiccation, it represented half a drachm (1.78 g) or 15% of the venom. Clinically, cobra venom was used at dilutions as low as 1:1000 (10⁻³). The volumes or quantities administered were quite small, often consisting of a pill or a drop of the diluted tincture. However, side effects associated with this form of administration caused the characteristic irritated and sore throat associated with cobra venom administration.

In conventional Western medicine, a defined quantity of a drug is administered to a patient in need thereof. For example, 1 mg of a drug can be given in a volume of 1 ml or 10 ml. In homeopathic medicine, however, the same volume of material is administered, regardless of the dilution factor. Furthermore, the dilution factors that used are quite large, ten-fold at a minimum, and more commonly 100-fold or 1000-fold in order to reach the exceedingly high dilutions that are routinely used by homeopathic doctors.

In the 1800's, it would not have been feasible to accurately weigh out the minute quantities of drugs that ended up in the final dilutions given to patients. The only way to obtain such small amounts of the active ingredient was to make progressive ten-fold dilutions, starting with a stock solution of known (high) concentration, and repeatedly diluting it. For purposes of comparing the dosages of venom in formulations described in the homeopathic literature of the 1800's with venom solutions of today (which are described in terms of protein concentrations (e.g. in mg/ml or μg/ml), the inventor first measured the protein content of raw cobra toxin as it is derived from the snake, and determined it to be about 350 mg/ml. With this information in hand, it was possible to estimate the approximate protein concentration that would be present in a homeopathic remedy prepared from raw venom, in which the starting solution (i.e., “mother tincture”) has a protein concentration of approximately 350 mg/ml (Table 1).

TABLE 1
Approximate protein concentration in dilutions of cobra venom used in
homeopathic formulations.

| Homoeopathic Formulation | Dilution | Protein Concentration | |
|---|---|---|---|
| Mother Tincture (0X) | 0 | 350 | mg/ml |
| 1X | 1:10 | 35 | mg/ml |
| 2X | 1:100 | 3.5 | mg/ml |
| 3X | 1:1,000 | 0.35 | mg/ml |
| 4X | 1:10,000 | 0.035 | mg/ml |
| 5X | 1:100,000 | 0.0035 | mg/ml |
| 6X | 1:1,000,000 | 0.00035 | mg/ml |

Serial dilutions such as these did not provide for dilution of cobra venom by a factor of 2× or 5×. It occurred to the inventor, however, that intermediate dilutions, i.e., greater than undiluted (referred to as “mother tincture” in the homeopathic literature) but less than ten-fold, might be very useful, if they provided the advantage of arriving at a dose that is therapeutically effective, safe, and lacking in the unpleasant side effects discussed above.

With the goal of achieving a standard dose of the active ingredient, the inventor produced a therapeutically effective (3×) homeopathic dose (e.g., 0.35 mg in 1 ml, if starting with a mother tincture having a concentration of 350 mg/ml) that could be administered in dosages such as a single dose in a 10-fold greater volume, or as 10 4× doses, or as 100 5× doses. Use of these dilutions might reduce the possibility of experiencing the adverse effects that caused this medication to be abandoned as a potent orally-administered analgesic. Tests were therefore conducted as described in the Examples below, using several liquid formulations based on homeopathic doses ranging from 2× to 5×. Results of clinical testing with human subjects with back pain showed effective reduction of back pain using dilutions of cobra venom stock solutions corresponding to each of these homeopathic doses. Positive results were achieved with a minimum of side effects reported previously using other dosage ranges.

From the standpoint of modern drug manufacturing, formulations in the 2× to 5× range were shown to provide several advantages over prior art formulations. For example, preparing the venom as a diluted liquid solution facilitates the preparation of a sterile product, and easier handling by automated systems.

The addition of a suitable edible preservative permits the sterile solution to be dispensed into containers for long-term storage and prevents the solution from becoming adulterated during the period of use. A metered spray or jet permits the venom to be administered as a controlled dose that allows frequent administration with limited esophageal irritation. The formulation and metered dose permits the venom solution to be administered over periods of days to weeks. These formulations may be useful for pain relief in both humans and/or animals.

The inventor has also discovered that a certain degree of effectiveness appears to accompany administering the venom to the mucous membranes of the mouth, such as would be achieved by an oral spray. This may also be accomplished, along with the additional benefit of potentially providing modified release compositions, using edible films such as those known to those of skill in the art. Such films and dissolvable strips have been made, for example, using pullulan, whey proteins, and other carbohydrates and proteins known to those of skill in the art of pharmaceutical formulation and administration.

The invention may be further described by means of the following non-limiting examples.

### EXAMPLES

### Example 1

### Oral Administration of a 5 mg/ml (“Homeopathic 1×”) Sterile Cobra Venom Liquid in Subject with Chronic Back Pain

From a stock solution of 400 mg/ml of sterile filtered cobra venom, a dilution was prepared by suspending 0.125 ml (50 mg) of the stock solution in 10 ml of saline, to reach a final venom concentration of 5 mg/ml. No secussion was required, because it was a final dilution.

This dilution is designated herein as a “homeopathic 1×.” However, it is to be noted that the terminology used in this context is not precise with respect to concentration, but rather covers a range of concentrations, because of the historical absence of dilutions of less than tenfold. For example, in classic homeopathy, starting with a mother tincture having a 350 mg/ml protein concentration (a modern concept not known or incorporated into homeopathic calculations), there is no intermediate dilution between 1× (corresponding to 35 mg/ml) and 2× (corresponding to 0.035 mg/ml). As used herein, any solution having a protein concentration in the range between 35 mg/ml and 0.35 mg/ml would described in homeopathic terminology as a “1×” homeopathic formulation because fractions (e.g., 1.1×, 1.2×, etc. are not used in homeopathic designations. However, as noted throughout the Examples, actual protein concentrations present in the samples are also provided.

A subject with chronic back pain was administered the 1× product prepared as described above, suspended in 10 ml of saline, by mouth. The reported taste was very unpleasant, provoking lacrimation and coughing. The unpleasant aftertaste persisted for some time, accompanied by a slight feeling of nausea, which may have been due to drinking the saline, rather than being attributable to the venom. The subject noted that his throat was tender and had a scratchy feeling similar to that of a sore throat treated with a numbing agent. The subject reported that stiffness in the back was noticeable, but not back pain. The subject also noted eyelids feeling heavy. A slight headache was noted 90 minutes after ingestion of the 1× solution that persisted for 8 hours. Throat symptoms were reported to be back to normal after 4 hours. No intestinal disturbances were reported.

### Example 2

### Oral Administration of a 0.333 mg/ml (“Homeopathic 3×”) Sterile Cobra Venom Liquid to a Subject

From a stock solution of 350 mg/ml of sterile filtered cobra venom, an aliquot of 0.143 ml (50 mg) was suspended in 10 ml of purified water (final concentration 5 mg/ml) with 1 minute of secussion. This solution was mixed with 140 ml of pure orange juice giving a final concentration of 0.333 mg/ml (equivalent to a homeopathic 3×). The formulation as described was administered to a subject with chronic back pain.

Upon ingestion, the subject reported no adverse effects save for a minor taste sensation in the mouth. None of the side effects traditionally associated with oral venom ingestion—such as esophageal irritation, lacrimation, coryza (acute inflammation of the mucous membrane of the nasal cavities; head cold), or intestinal disturbances were reported. Importantly, the same amount of venom drug was delivered (i.e., 50 mg) as in Example 1 above, however the volume of liquid was much greater (150 ml vs. 10 ml.) This result indicated that the same dosage (50 mg venom protein) that previously caused unacceptable side effects was completely tolerable when delivered in a much higher volume of liquid.

### Example 3

### Oral Administration of a 1 mg/ml (“Homeopathic 2×”) Sterile Cobra Venom Liquid in a Subject with Chronic Back Pain

The subject with chronic back pain of Example 1 was administered 0.0125 ml (5 mg) of a 400 mg/ml mother tincture of sterile filtered cobra venom solution, taken orally in ml water (final concentration 1 mg/ml). No secussion was required because it was a final dilution.

In this example, a 1× solution would contain 40 mg/ml protein, a 2× solution would contain 4 mg/ml protein, and a 3× solution would contain 0.4 mg/ml protein. The final solution given to the patient, having an intermediate concentration of 1 mg/ml, which is less than 2× (4 mg/ml) but greater than 3× (0.4 mg/ml) is designated as being equivalent to a homeopathic 2× or 1C).

At the time of administration, the subject's back pain was estimated to be 4-5 on a scale of 1-10. The patient reported that the taste of the diluted solution (as compared with a 1× solution described in Example 1) was not nearly as harsh as before, even with the solution being rinsed around in the mouth before swallowing. The subject reported, however, that the taste worsened over time. Ninety minutes after administration, the patient reported a pain level of 0.5-1, with no adverse effects. Second and third administrations of a 5 mg dose at 8 and 24 hours after the first administration resulted in no adverse responses.

### Example 4

### Oral Administration of a 0.4 mg/ml (“Homeopathic 3×”) Sterile Cobra Venom Liquid in a Subject with Chronic Back Pain

A subject, while experiencing back pain at a level of 3-4 on a scale of 10, was administered an oral cobra venom product prepared as described in Examples 1 and 2 above, but using a 3× formulation (0.4 mg/ml) in water. More specifically, the oral formulation was prepared by using 0.02 ml from a mother tincture of 400 mg/ml (aliquot thus containing 8 mg venom). The aliquot was diluted into 20 ml of purified water, yielding a 3× formulation with a final venom concentration 0.4 mg/ml.

The subject noted taste deterioration, and throat sensations as described above. A dull headache was noted 90 minutes later. Back pain was reduced to 1 to 1.5. Twelve hours later, the subject reported that the headache persisted, but the backache was reduced to a pain level of 0.5 on a scale of 1-10. Second and third doses of the 3× formulation were taken on the second and third days, respectively. No adverse effects were noted, including absence of the characteristic throat irritation associated with previous homeopathic formulations of cobra toxin. Back pain levels were reported to be less than 0.5. No gastrointestinal upset was experienced.

### Example 5

### Oral Administration of a 0.07 mg/ml (“Homeopathic 4×”) Sterile Cobra Venom Beverage in a Subject with Chronic Back Pain

The subject in this Example was experiencing back pain at a level reaching 7-8 upon standing, and settling in around 5. A beverage of cobra venom was prepared by adding 0.01 ml (3.5 mg) of a 350 mg/ml sterile cobra venom “mother tincture” solution to purified water containing 5% pure lime juice, and 0.2% citric acid, made up to a final volume of 50 ml. Final concentration of the venom in the beverage solution was 0.07 mg/ml. Having a final concentration greater than 5× (0.0035 mg/ml), but less than 4× (0.035 mg/ml), this formulation would be designated as within the range of a homeopathic 4× solution.

The subject reported minor irritation to the throat, although it was deemed to possibly have been attributable to either the venom or the lime juice. Within 1 hour, the subject's pain level was reduced to 3-4, and the subject could stand up and sit down easily. The subject was also able to touch his toes easily, which was usually not the case, suggesting some relaxation of muscles. Seven hours post administration, the pain level was further reduced to a level of 2-3. The subject also reported a significant improvement in sleep quality. Notably, the characteristic headache, typically experienced with the liquid formulations as described in Examples 1-3, failed to appear after ingestion of the beverage formulation. No gastrointestinal upset was experienced.

### Example 6

### Pain Relief Product Comprising Flavored 0.035 mg/ml (“Homeopathic 4×”) Sterile Cobra Venom Solution in an Oral Spray Dispenser

A formulation of sterile cobra venom at “4×” with citric acid, flavoring and methyl paraben was prepared. To do so, a 2× dilution was made from a mother tincture solution of 350 mg/ml (2×=3.5 mg/ml). To produce a 4× solution, the 2× solution was diluted 1:100, to produce a solution with a final venom concentration of 0.035 mg/ml. The formulation was filled into bottles to a volume of 20 ml (final concentration of venom 0.035 mg/ml). In this case, the final protein concentration (0.035 mg/ml) corresponded precisely to that calculated according to Table 1 for a homeopathic 4× formulation. Bottles were fitted with a pump dispenser configured to deliver 0.1 ml of solution per actuation.

Over the course of two weeks, several such dispensers comprising the flavored sterile cobra venom composition were used to deliver two sprays, four times per day. Each spray dose of 0.1 ml volume contained 0.0035 mg of venom protein. Accordingly, a two-spray dose would deliver 0.007 mg venom per application). Use of the spray as directed (two sprays, four times per day), thus delivers a total dose of 0.028 mg of venom per day.

At two weeks, a sterility test was conducted on the contents by spreading a 0.5 ml volume of the product onto bacterial agar plates. The results showed no growth was observed following three days of incubation.

### Example 7

### Oral Administration of 0.035 mg/ml (“Homeopathic 4×”) Sterile Cobra Venom by Oral Spray in Subjects with Chronic Back Pain

A formulation of sterile cobra venom at 4× homeopathic concentration as described in Example 5 was prepared and packaged in pump dispensers. The product was provided to six subjects with various types of chronic pain, with instructions to administer two sprays every 3-4 hours daily.

In general, a satisfactory reduction in pain was achieved in over 70% of the subjects. No gastrointestinal upset was reported, although a minor esophageal irritation was experienced, described as a dryness which diminished with continued use. In no case was the esophageal irritation sufficiently uncomfortable to discourage continued use of the pain relief product.

### Example 8

### Oral Administration of a 0.07 mg/ml (“4×”) Sterile Cobra Venom by Oral Spray in Subjects with Chronic Back Pain

A formulation of cobra venom at 4× with citric acid, flavoring and methyl paraben was prepared in the manner described in Example 5 and packaged in pump dispensers. In this case, however, the final concentration of venom in the spray formulation was 0.07 mg/ml. If fractions were permitted, this formulation could be described as equivalent to a “3.8×” homeopathic formulation, as compared with the 4× formulation of Example 7.

This product was provided to 20 subjects with various types of chronic pain, who were instructed to take two sprays every 3-4 hours daily. In this group as well, a satisfactory reduction in pain was achieved in over 70% of the subjects. No gastrointestinal upset was reported, although minor esophageal irritation was experienced, as noted by subjects described in Example 6.

### Example 9

### Oral Administration of 0.175 mg/ml (“4×”) Sterile Cobra Venom by Oral Spray in Subjects with Chronic Back Pain

A formulation of sterile cobra venom was prepared as described above, this time at the protein concentration of a homeopathic “3.5×” as defined in Example 8. Final concentration of venom in this formulation was 0.175 mg/ml. The spray formulation included citric acid, flavoring and methyl paraben, and was packaged in pump dispensers.

The product was provided to eight subjects with various types of chronic pain. Patients were instructed to take two sprays every 3-4 hours daily. In this instance, a satisfactory reduction in pain was achieved in over 90% of the subjects. Improved pain response was noted over the previous 4× formulation. No gastrointestinal upset was reported, although minor esophageal irritation as described above was observed. Nevertheless, this irritation was not severe enough to cause the subjects to discontinue use of the product.

What is claimed is:

1. An analgesic composition comprising a sterile cobra venom solution admixed with a food-grade preservative in a metered pump configured to deliver the composition as an oral spray or jet.

2. The analgesic composition according to claim 1, wherein the sterile cobra venom solution has been sterilized by filtration through a filter having a pore size of about 0.45 μm or less.

3. The analgesic composition according to claim 1, wherein the sterile cobra venom has been sterilized by prolonged low-heat treatment or by pasteurization.

4. The analgesic composition according to claim 1, wherein the sterile cobra venom solution has a final homeopathic formulation of 3×.

5. The analgesic composition according to claim 4, wherein the protein concentration of the sterile cobra venom solution is from about 0.035 mg/ml to about 0.35 mg/ml.

6. The analgesic composition according to claim 1, wherein the sterile cobra venom solution has a final homeopathic formulation of 4×.

7. The analgesic composition according to claim 6, wherein the protein concentration of the sterile cobra venom solution is from about 0.0035 mg/ml to about 0.035 mg/ml.

8. The analgesic composition according to claim 1, wherein the sterile cobra venom solution has a final homeopathic formulation of 5×.

9. The analgesic composition according to claim 8, wherein the protein concentration of the sterile cobra venom solution is from about 0.00035 mg/ml to about 0.0035 mg/ml.

10. A method for the alleviating pain in a human and/or animal subject, the method comprising orally administering a composition comprising a sterile cobra venom solution admixed with a food-grade preservative, the venom solution having a homeopathic formulation of from about 3× to about 5× and the step of administering being performed using a metered pump configured to deliver the composition as an oral spray or jet.

11. The method according to claim 10, wherein the metered pump delivers a volume of solution in the range of 0.05 ml to 1 ml.

12. The method according to claim 11, wherein the metered pump delivers a volume of solution in the range of 0.1 ml to 0.2 ml.
